(19) United States
(12) Patent Application Publication
(10) Pub. No.: US 2002/0193693 A1
(43) Pub. Date: Dec. 19, 2002

(54) Method of analysis of cardiovascular condition

(76) Inventor: Alexander Kramarenko, Kharkov (UA)

Correspondence Address: Ilya Zborovsky, 6 Schoolhouse Way, Dix Hills, NY, 11746, US

(21) Appl. No.: 09/882,966

(22) Filed: Jun. 18, 2001

(51) Int. Cl. A61B 5/04

(52) U.S. Cl. 600/509

(57) ABSTRACT

A method of analysis of cardiovascular condition, includes generating an electrocardiogram of a heart activity of a patient; and analyzing the generated electrocardiogram by determining a magnitude of a red shift of a signal spectrum.

### BACKGROUND OF THE INVENTION

The present invention relates to method of analysis of cardiovascular conditions.

It is known to determine heart conditions or generally conditions of a cardiovascular system by generating electrocardiogram and analyzing the latter.

The spectroanalysis of an electrocardiogram can be performed with various methods. In a traditional method of obtaining of an energy spectrum, a main harmonic is a periodical oscillation, which changes in frequency, amplitude and shape of complexes, determined by electrical phenomena or contraction of a cardiac muscle as shown in FIG. 1. Due to a sufficiently great (more than one period) of realization, a frequency resolution is high and depends, in accordance with the principle of indetermination, from a length of realization, with a frequency of a main harmonic determined by a cardiac rhythm, or in other words by a frequency of heart contractions, and its amplitude is determined by an average amplitude of electrocardiogram, while its width along an axis x is determined by a frequency deviation of the signal. The energy of various peaks and segments of the electrocardiogram is transferred to higher harmonics, in accordance with the simplified interpretation. While the obtained spectrum is a correct transformation of a signal from a time area to a frequency area, monitoring of (changes of electrocardiogram within the given realization is impossible. In addition the frequency modulation caused by constant change of frequency of heart contractions does not make possible the evaluation of a true width of main part of the spectrum. Its harmonics and inaccuracies of spectral evaluation in form of energy transfer make difficult the evaluation of low amplitude components, which represent a result of conversion of peaks and intervals of the electrocardiogram, which is a primary interest of a cardiologist.

### SUMMARY OF THE INVENTION

Accordingly, it is an object of the present invention to provide method of analysis of heart condition, which can be considered as more accurate than the existing methods.

In keeping with these objects and with others which become apparent hereinafter, one feature of present invention resides, briefly stated in method of analysis of heart condition which comprises the steps of generating an electrocardiogram of a patient; and analyzing the generated electrocardiogram by determining a magnitude of a red shift of a signal spectrum.

In accordance with one embodiment of the present invention said determining includes using a relationship of areas of high frequency and low frequency strips of the spectrum.

In accordance with another embodiment of the present invention said determining includes using a sine of an angle of inclination of a straight line which connects centers of rectangles whose areas are proportional to a power in a high frequency strip and a low frequency strip of the spectrum.

### BRIEF DESCRIPTION OF THE DRAWINGS

FIG. 1 is a views schematically showing an electrocardiogram;

FIG. 2 is a view illustrating an electrocardiogram for a patient which has a heart attack;

FIG. 3 is a view showing a compressed spectral area of the electrocardiogram demonstrating a red shift;

FIG. 4 is a view showing a shape of curves during the process of myocardial infarction;

FIG. 5 is a view showing a short term restoration of a sinus rhythm in a patient with a post cardiosclerosis;

FIG. 6 is a view showing a shape of the curve of an electrocardiogram of an electrocardiogram of another patient;

FIG. 7 is a view showing a curve of an electrocardiogram for a healthy person;

FIG. 8 is a showing an electrocardiogram during an electrocardiogram during a test with a physical load;

FIG. 9 is a view illustrating a dependency of a red shift of a spectrum from a frequency of cell pulses;

FIG. 10 is a view showing an electrocardiogram with single extra systoles;

FIG. 11 is a view showing an electrocardiogram for abnormal heart contractions;

FIG. 12 is a view showing an electrocardiogram during thrombolic therapy of acute myocardial infarction;

FIGS. 13-15 are views showing curves of red shift in accordance with the present invention; and

FIG. 16 shows a spectrum corresponding to the inventive method.

### DESCRIPTION OF THE PREFERRED EMBODIMENTS

The present invention deals with a method of determination of cardiovascular conditions, such as heart conditions with the use of an electrocardiogram.

Dynamics of the electrocardiogram in particular in the case of long-term Holter monitoring, can be evaluated with the method of compressed spectral areas. The spectra even for short realizations have the same disadvantage, which is caused by the presence of a powerful leaf which is shifted in frequency. It is therefore advisable to select a sliding window of a minimal size and to average the data inside each realization, which represents a single element of compressed spectral areas. This, in addition, will make possible to reduce requirements to a computing device. In the present invention, a sliding window with a length of one second is selected for each realization with the length of one minute. With this approach the main leaf with its unpredictable frequency modulation and high energy can be eliminated.

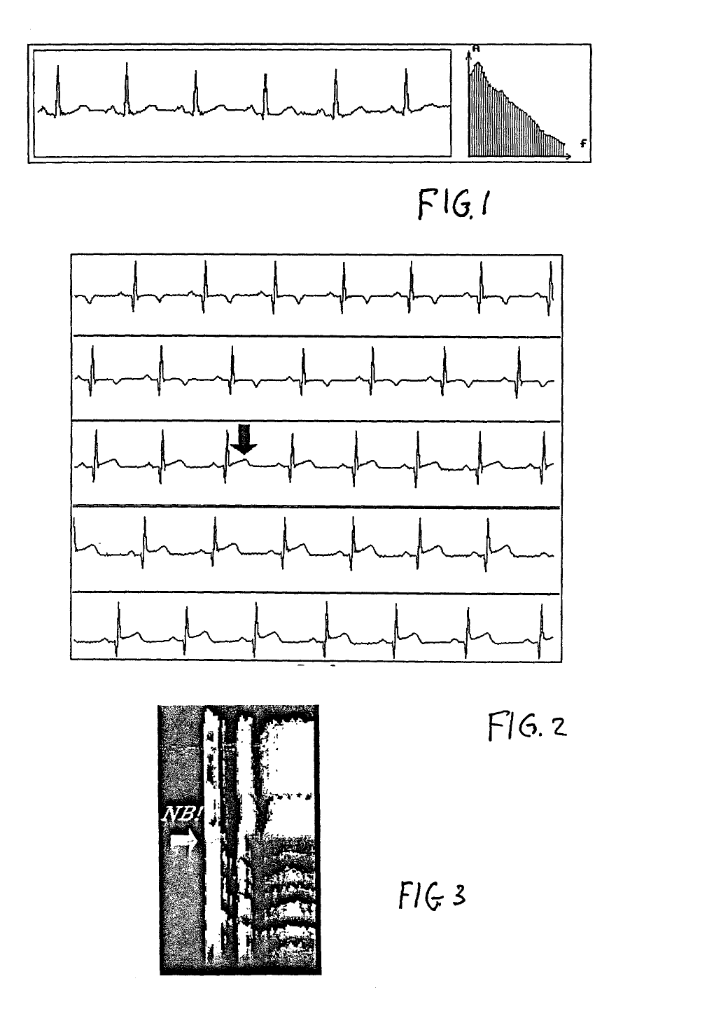

With this approach it is possible to register a hypothetical red shift. It is assumed that this phenomenon occurs in moments of discreditation of a system of blood supply. Various types of pathology do not have to be considered. The main requirement is to have a visual change in the process of registration of electrocardiogram, or in other words when such changes do not occur for a certain time, and then they develop. FIG. 2 shows exactly this case. A patient developed a relapsed myocardial infarction which can be easily seen from a shift of a segment ST. The process is developed acutely, during few minutes.

Compressed spectral areas demonstrate a typical red shift, which is expressed as a shift of all spectral components to the left, and also in the form of redistribution of energy into a low frequency area of the spectrum as shown in FIG. 3.

In order to evaluate a magnitude of the red shift it is possible to use two parameters which are close to one another. One parameter is a sine of an angle of inclination of a spectrum, while the other parameter is a ratio of a low frequency and a high frequency strips expressed in decibels. A more traditional method of calculation of a median frequency of a spectrum proved to be not efficient, which is caused probably by a non-standard approach to a spectrum evaluation.

FIG. 4 shows how in a moment of relapse of myocardial infarction both curves started raising and then stopped in a future at the high level. It is necessary to pay attention to the fact that a short-time raise of the curves appeared before development of infarction, and the shape of complexes EKG during this time did not have yet visually seen changes. It is possible that this is connected with an insufficiency of single-channel registration, since the changes were present, but in this channel they appeared weekly, invisibly for an eye.

Infarction is a root and distinct pathology of a heart muscle, which cause catastrophic (in a mathematical sense) changes in a functioning of the whole system. Therefore the phenomena of a red shift has to be confirmed by observations of pathologist which are not so pronounced.

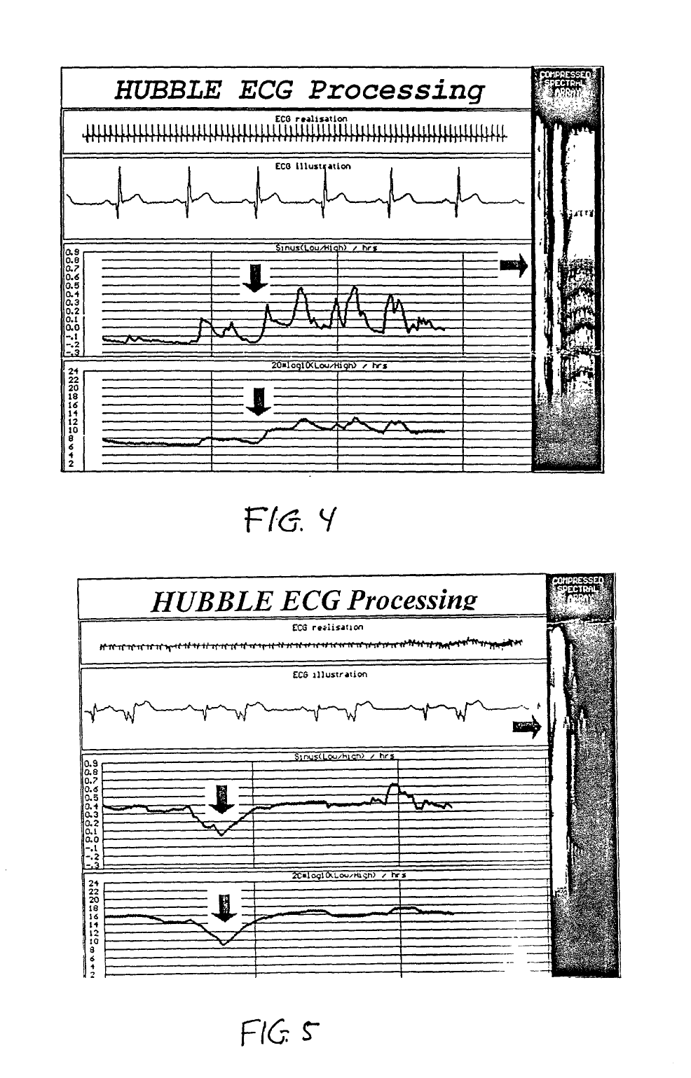

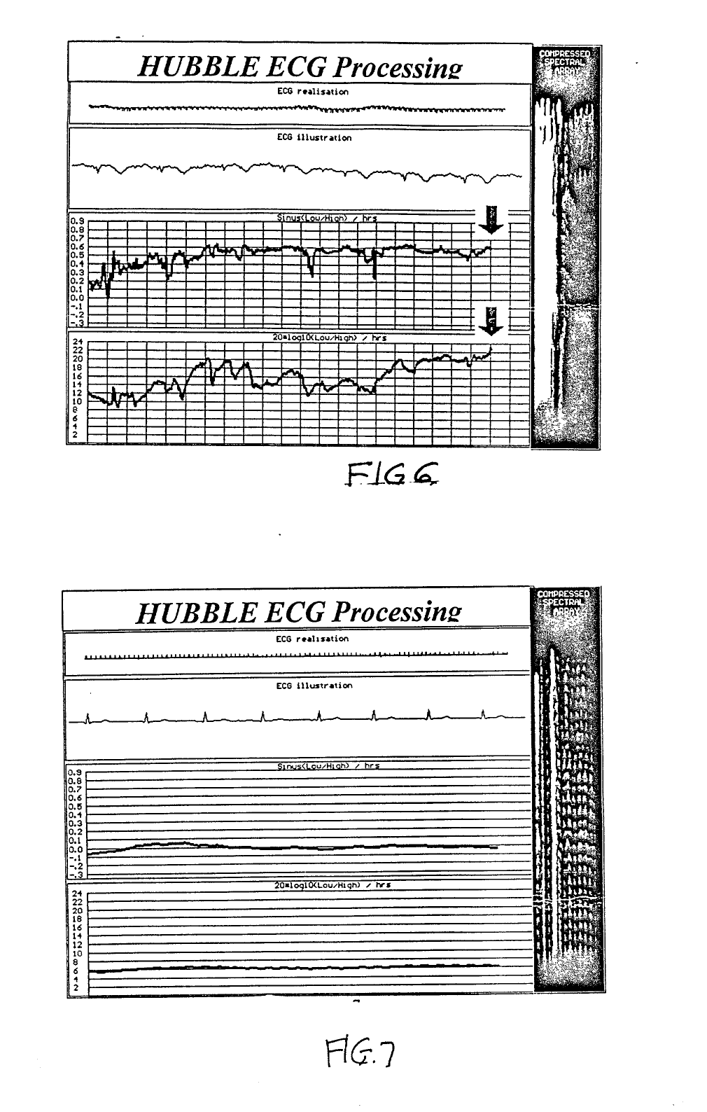

FIG. 5 shows a (case of a short term restoration of a sine rhythm in a patient with a post-infarction cardiosclerosis.

It is necessary to pay attention to the fact that in addition to a restoration of sine rhythm nothing catastrophic occurred. Nevertheless both parameters change significantly, which is a non obvious result. Actually if the red shift in the cases of significant deformation (expansion) of cardiocomplexes can be forecast during a visual evaluation of an electrocardiogram its reduction during normalization of a rhythm will meet a physical and a biophysical explanation. This is why it is important to investigate a case of a catastrophe with a fatal consequence for the whole system and organism as a whole. During this process a magnitude of a red shift must symptomatically approach a certain critical level and reach this level. In particular in this moment a catastrophe with a fatal consequences must occur.

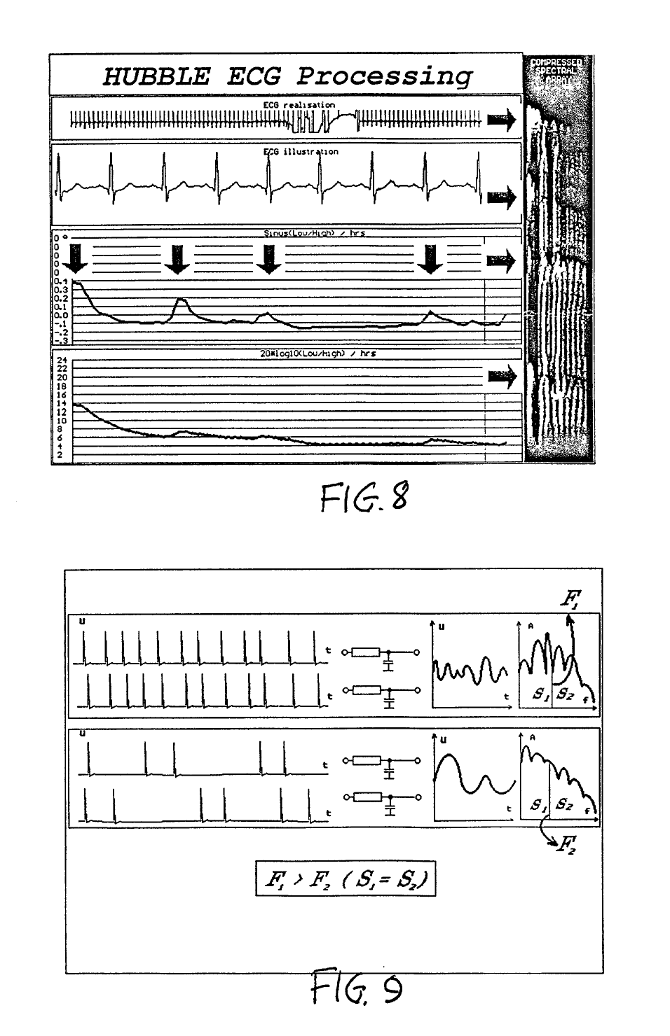

This case is considered in detail and illustrated in FIG. 6. Patient C 63 years old was admitted to a cardiological department because of acute myocardial infarction. Despite of complex treatment, the condition of patient remained grave, angina pains occurred again, the conditions become aggravated with paroxismal shape of fibrillation of heart and extra systolic arrhythmia. On 8th day of patient's stay in a hospital, its condition suddenly worsened and a clinical death occurred which was caused by fibrillation of ventricles. Reanimation steps did not succeed. In one hour a biological death was diagnosed. On the section an acute trasmural front-partition-upper myocardial infarction with a formation of acute aneurism with a wall-adjacent clot was diagnosed. The cause of death was a linear tearing off of a front wall of the left ventride with a hemotamponage of heart. Attention should be paid to very high and growing values of two above mentioned parameters. They were not registered in any observation of patients who survived. For healthy test patients both parameters are retained stably in the areas sin (a):−0.1 . . . +0.2 and L/H: 4. . . 8 db as shown in FIG. 7. F.

It is necessary to pay attention to the fact that when tests with a physical stress were performed, both of these variables grow as shown in FIG. 8. This fact is unobvious-a frequency of heart contractions increased, the shape of the electrocardiogram complex however practically did not change, while a red shift grows.

It is believed to be desirable to present some statistic data for each group of pathologists.

A first consideration is that in the case of any alteration a cell which generates a pulse electric, signal (neuron, cardiomicyte, etc.) reduces a limiting frequency of generation of pulses, and in the case of a more significant damage there amplitude reduces as well. Since the output signal is a single-pole pulse sequence, it can be demodulated by an integrator, as well as a width-pulse modulated. From this fact a principle and very important conclusion can be made, that during propagation to a surface of a three-dimensional conductor (body surfaces) which has its own active resistance and capacity, a cell signal will be partially demodulated by an integrator formed by passive RC chains of surface tissues. This mechanism usually is not taken into consideration during explanation of a cause of a surface electro-physiological signal (only summation is considered). Therefore, the dependency of a value of red shift of a spectrum of a sum signal from an extreme frequency of sequence of cell pulses is a function of passive RC chains, or in other words in a variant in time system. FIG. 9 illustrates this mechanism.

A conclusion which can be made is that, as soon as in a group of cells which spontaneously generates pulse sequences, a fraction of damaged cells which operate with a lowered extreme frequency is increased, then in a spectrum of a signal registered on a body surface a red shift appears. This explanation can be acceptable for the cases of myocardial infarction, cardiomeopathy, myocarditis and other pathologies, which is accompanied by damages of cells. In addition, this mechanism is a main mechanism during a lowering of a median frequency of a spectrum of electroencephalographic signal in the cases of rude brain pathology. For observations which register phenomena of red shift during disturbances of a cardiac rhythm, it does not make any contribution or its influence is secondary.

A second, in the vase of an electrocardiogram and might be a first one, there can be a principle of discordination of a generator with a load. A mechanical generator of heart must be coordinated with a vascular system, or otherwise efficiency of its work can approach zero. For example, reduction of an opening of aorta causes discordination of the whole system only because, in a period of heart expulsion it is not possible to pump a required quantity of blood. In order to eliminate this defect, it is necessary either to increase a pressure of expulsion or to increase a length of a phase of expulsion. Both these approaches can be possible only to a certain limit, which is determined by mechanical characteristics of heart. The system of course must be coordinated with many parameters-hydrodynamic resistance, resonance frequency, etc. It is to be understood that in a space of parameters there is certain optimum within which the system has a maximal efficiency. Beyond these limits the coordination worsens and load on a heart muscle grows not proportionally to a required volume of pumped blood, but significantly faster (by a function of dis coordination). A closer and very good analog can be a system of a transmission-antenna-feeder device, where efficient operation of the antenna is determined not only by its amplification and power of a transmitter, but also by a quality of coordination of an output cascade, line and antenna. If such system operates beyond the limits of acceptable frequency range with unacceptable coefficient of a standing wave, only an overheating of a transmitter and even its breakage can occur.

In the cardiovascular system, a sample of discoordination can be a ventricle extra systole. Its spectrum is significantly shifted to a low frequency area, its amplitude is significantly higher than in a normal systole, a quantity of time of expelling is greater. As a result mechanical energy does not propagate into a vessel mouth, but instead is dispersed in a heart muscle.

This is another important question: why the dispersed electrical energy of electrosystole is much greater when compared with a normal heart contraction. It is not possible to find a response in the existing explanation of a cause of electrocardiogram. The quantity of reduced cardiocytes is equal to the quantity at the normal contraction, despite of a different, longer path of propagation of the excitation wave. If the electrocardiogram is generated only by cardiocytes, then an increased of electro energy in extrasistola is impossible in principle. Thereby a hemodynamic component of generation of electrical signal becomes a reality. And this has some role for formation of a red shift in a spectrum of signal. An explanation for this can be as follows. A propagating valve of excitation in any moment of time forms an electrical dipole, whose orientation is determined by a stroke of excitation wave. Therefore if heart does not pump blood in this moment, a change of polarity of an electrical signal at the registering electrodes would be determined only by a change of orientation of this dipole, while a frequency of the changes would be determined by a frequency of change of orientation. In reality, the process is slowed by inertia of mass of blood which moves with a changing acceleration, which causes an increase of level of low frequency components of the process. The more efficiently operates the system, the greater quantity of blood moves along non-optimal trajectories inside heart chambers, which causes the dispersion of a greater quantity of energy, whose portion is converted into an electrical energy and leads to an increase of amplitude of the electrical signal. An increase of a displaced mass leads to slowing of the whole process, since the energy of reduction of cardio mycites remain the same. A simple and adequate model which explains an occurrence of a phenomena of red shift during a ventricle extra systole can be an explosion of a certain quantity of a matter. If a special explosive is used which initiates a detonations in a “cord” extended through the whole volume of a substance, the explosion is short and efficient. If an explosion goes only in one poin:, the time of propagation of an explosion wave over a whole volume is increased in efficiency of the explosion is reduced, while the energy is the same in both cases. A spectrum of “slow” explosion will be shifted to a low frequency area. During a normal heart contraction, an excitation propagates along a conductive system of “cord”, while a ventricle electro systole starts from one point and propagates by a wave.

The occurrence of a red shift during physical loads in a healthy person can be explained by the same mechanism of discoordination of a mechanical generator of heart with a load of a vascular system, or in other words the case of the situation when parameters extend beyond a range of a physiological optimum. It can be seen that the magnitude of a shift of spectral components will be always less than during a ventricle extrasystole and this is what is seen in reality. For above ventrile extrasystole the magnitude of the red shift will be greater in response to the greater difference of a current time interval from a preceding interval. The red shift of the upper ventricle extrasystole must be always less than ventricle, while for a post-extrasystole complex it also must exist (a small magnitude) despite its generally conventional shape.

It therefore can be concluded that:

1. Red shift is a universal parameter which corresponds to an inefficient operation of a cardiovascular system.

2. Any damage to a cardiac muscle increases the red shift, restoration of its function reduces the red shift.

3. Discoordination, or in other words reduction of reserve possibilities of cardiovascular system has a correlation with a magnitude of a red shift.

4. A quantitative evaluation of this phenomenan is usable for a diagnostics, in particular for determination of absolute magnitudes, and their tendencies in particular in the cases of long-term observation, such as Holter monitoring.

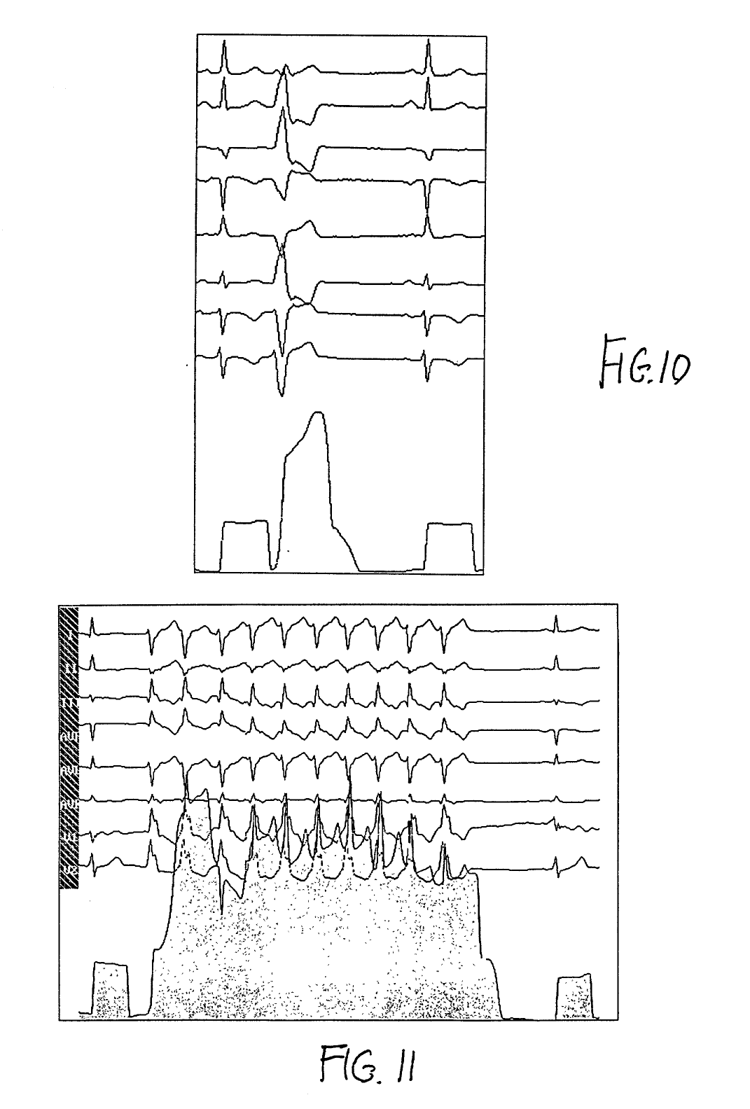

FIG. 10 shows 3 case of generating an electrocardiogram with single electrosystolas. A lower curve is a result of operation of integrator with a sliding window 400 m sec length. It can be seen that a total electrical energy of the extra systole several times greater than in the case of normal heart contraction. This as a result can lead to the following conclusion. Even if a contribution of a hemodynamic (component is zero for normal heart contraction, in the case of ventricle electrosystole, it exceeds many times the sum electrical energy generated by heart cells during a normal contraction.

It can be expected that in the case ventricle tachocardia, or in words in the case of abnormal heart contractions, an instantaneous electrical power and a sum electrical energy will be even higher than in the case of single ventricle electrosystole. This is observed in FIG. 11. Naturally in such cases an additional increase of some electrical energy can be caused by the mechanism of reentry.

It should be mentioned that in the cases of such episodes the red shift reaches certain extra critical magnitudes (greater in the patients before their death), which probably is caused by short-term overloading of the cardiovascular system, that becomes fatal when its time increases. This can be the reason why ventricle tachocardia is so dangerous.

It is important to present other examples. For example in sports medicine it is known to evaluate amplitude of differential electrocardiogram. The greater is amplitude, the higher potentials of a sportsman in the case of high physical loads. However, the amplitude of differential electrocardiogram is correlated with a magnitude of shift of spectral components. It is known that the time of propagation of wave of excitation through a miyocard is approximately 100 m sec. A spectrum of single rectangular pulse (sin(x)/x) is a good approximation with an expected spectrum of excitation wave. The generation of significant red shift can be cause only by an expansion of this pulse, or in other words by a delay of propagation of excitation wave. If in the case of pathologies a delay of propagation is not observed, but a red shift takes place. This can not be explained by electrical processes only in a miyocard. Moreover, the magnitude of red shift can allow an indirect evaluation of a contribution of components of electrocardiogram, which have nothing to do with a process of excitation wave.

Therefore it can be said that the red shift is not only a universal method of evaluation of function of cardiovascular system, but also a method of analysis of mechanism of generation of a surface electrocardiogram. This can be confirmed by FIG. 12 which shows monitoring of electrocardiogram during a thrombolic therapy of acute myocardial infarction. It can be assumed that after development of ischemic process some red shift must appear, which is determined by disturbances of hemodynamics and intra cardiac conductivity, but not by subjective gravity of clinical picture. As a result of a successful thrombotic therapy it must be reducing with a speed equal to the speed of restoration of function of a cardiac muscle. But the very moment of beginning of restoration of blood circulation in the area of ischemia must be accompanied by mechanical and electrical processes which are not correlated with the activity of index areas. In other words a transition to a normalization must be through short-term increase of the red shift, since chaotically contracting region of a cardiac muscle causes a more significant disturbance of hemodynamics than an immovable one. In FIG. 12 the introduction of thrombolic preparation coincides with the beginning of the recording. It is obvious that normalization of functioning of a heart will start only in a few days. Because of this five hours earlier only a partial reduction of both parameters shown in the graft was observed. However, the moment of thrombolisis with a short term increase of the red shift which took place about 20 minutes can be easily observed. In 48 hours both parameters still were slowly reducing.

It can be seen that the method of measurement of the magnitude of red shift is suitable for evaluation of efficiency of thrombolic therapy and the degree of danger of the moment of thrombolisis.

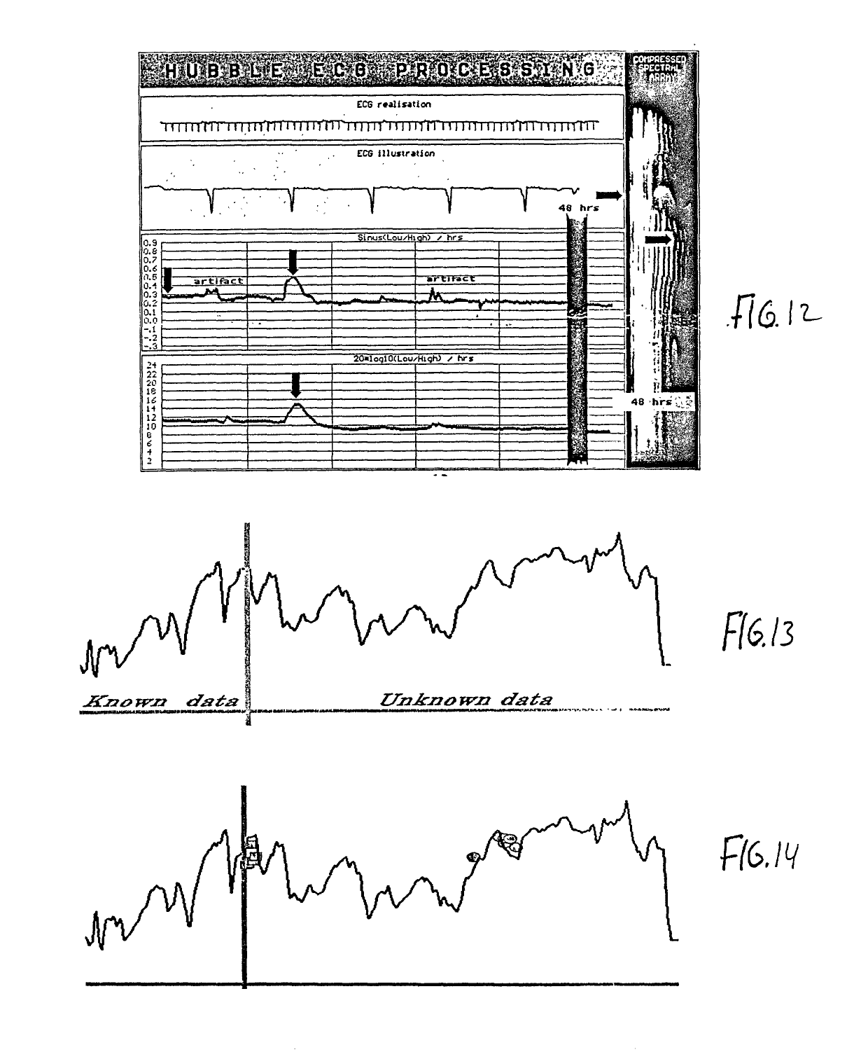

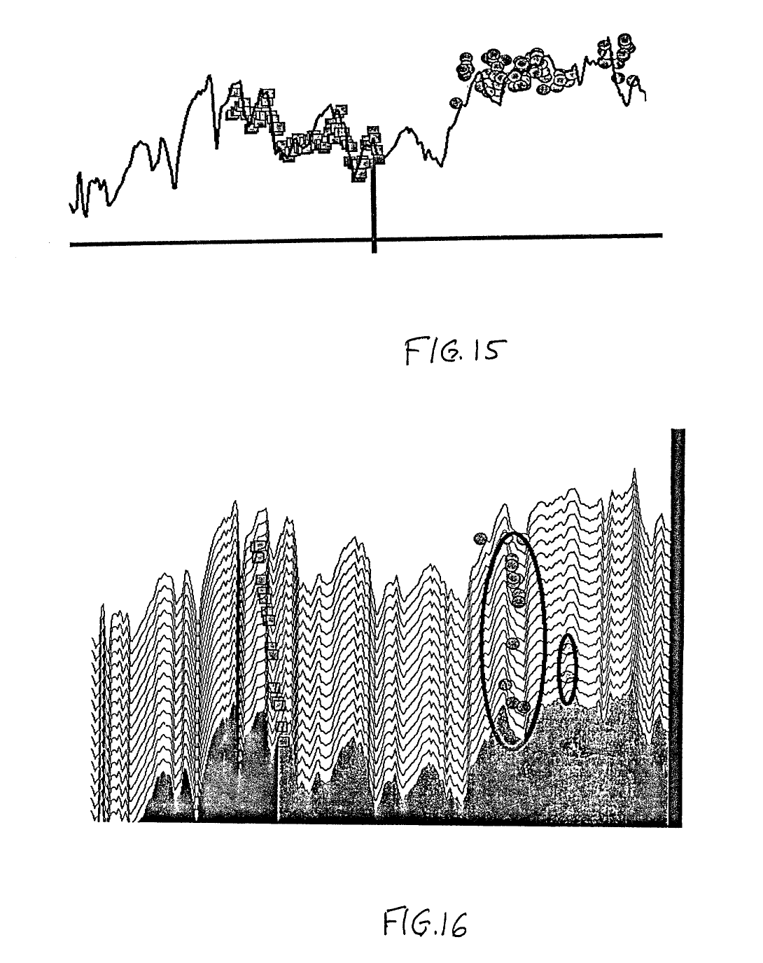

It is important to provide some additional explanation. Let us consider the case which is presented in FIG. 6. Assuming that the curve of the red shift is known only up to the moment (FIG. 13) let us make a short-term term forecast with a lead time of two minutes and a long term forecast with a search of a global extremum of the forecast curve. The results shown in FIG. 14 illustrate that the forecast is very accurate, and an error of short-term forecast is even higher than of the long term forecast. If the forecast is further continued with a sliding window of data, the results can be seen in FIG. 15. A low error of the forecast can be seen, which is very strange, since the results are too good to be true. In order to provide a control, the data with an additional development along the axis Y must be presented in time as shown in FIG. 16. The result is identical: a forecast of a raise of the curve was performed correct with the lead time 12 hours.

It is true it is believed to be clear that the system of forecast of the magnitude of red shift can present a significant interest for hospitals for evaluation of forecast as well as for evaluation of deviation of real data from forecast.

It will be understood that each of the elements described above, or two or more together, may also find a useful application in other types of methods differing from the types described above.

While the invention has been illustrated and described as embodied in method of analysis of heart condition, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A method of analysis of cardiovascular condition, comprising the steps of generating an electrocardiogram of a heart activity of a patient; and analyzing the generated electrocardiogram by determining a magnitude of a red shift of a signal spectrum.

2. A method as defined in claim 1 wherein said determining includes using a relationship of areas of high frequency and low frequency strips of the spectrum.

3. A method as defined in claim 1, wherein said determining includes using a sine of an angle of inclination of a straight line which connects centers of rectangles whose areas are proportional to a power in a high frequency strip and a low frequency strip of the spectrum.
